(19) United States
(12) Patent Application Publication
(10) Pub. No.: US 2021/0280285 A1
(43) Pub. Date: Sep. 9, 2021

(54) SYSTEM, DEVICE AND METHOD FOR MEDICATION COMPLIANCE MANAGEMENT

(71) Applicant: PillTracker Ltd., Tel Aviv (IL)

(72) Inventors: Zachary Javitt, Tel Aviv (IL); Jonathan Javitt, Tel Aviv (IL); Chuan Wang, Tel Aviv (IL); Jun Ouyang, Tel Aviv (IL)

(21) Appl. No.: 16/334,455

(22) PCT Filed: Sep. 15, 2017
(86) PCT No.: PCT/IB2017/001301

§ 371 Date: Mar. 19, 2019

Related U.S. Application Data
(60) Provisional application No. 62/396,338, filed on Sep. 19, 2016.

(51) Int. Cl. G16H 20/10; G16H 40/20; G16H 40/67; G16H 10/60; G06K 1/12; G06K 19/067; G06Q 30/00; H04L 29/06; A61J 1/03; B41M 5/00

(52) U.S. Cl. CPC G16H 20/10; G16H 40/20; G16H 40/67; G16H 10/60; G06K 1/121; G16H 10/20; G06Q 30/0185; H04L 63/08; A61J 1/035; B41M 5/0023; G06K 19/067

(57) ABSTRACT

A system for tracking medication compliance includes a blister pack, a blister pack tray, an end user computing device, and a communications cloud. The blister pack is modified with conductive ink for transmitting data relating to medications stored in the blister pack. The blister pack tray is adapted to receive the blister pack and to receive data transmitted from the blister pack. The end user computing device is adapted to mechanically connect to the blister pack tray, whereby the end user computing device is configured to receive data relating to medications stored in the blister pack, and wherein the computing device is configured to run software to identify changes in the status of medications stored in the blister pack. The communications cloud includes a medication compliance tracking server and a medication compliance tracking database.

### CROSS-REFERENCE TO RELATED APPLICATION

This is an international (PCT) application relating to and claiming the benefit of commonly-owned, copending U.S. Provisional Patent Application No. 62/396,338, filed Sep. 19, 2016, entitled “SYSTEM, DEVICE AND METHOD FOR MEDICATION COMPLIANCE MANAGEMENT,” the contents of which are incorporated by reference herein in their entirety.

### FIELD OF THE INVENTION

The present invention relates in general to systems, methods and devices useful in medication management, tracking, and compliance,

### BACKGROUND OF THE INVENTION

It has become common in recent years for medication consumers to use digital means, and in particular, smart phone type devices, to help them track their medication consumption. Today there are many applications or software for usage on smart phones or wearable devices, to help users note medication conception, as well as alert users and their selected social networks of such consumption or lack of conception.

However, the user management of their actual medications still provides substantial problems for users, leading to problems in medication compliance and many other negative consequences. For example, consumers of multiple medications, as well as young and elderly consumers may be more challenges to keep in control of the ordering, collecting, storing, preparing, and correctly consuming their medications.

It would be highly advantageous to have a system or method that could enable medication consumers to manage medication procurement (ordering and retrieval) and consumption, to facilitate enhanced compliance.

### SUMMARY OF THE INVENTION

There is provided, in accordance with an embodiment of the present invention, an apparatus, system, and method to provide, enhanced medication management, tracking and compliance.

According to some embodiments of the present invention, a system is provided tracking medication compliance, that includes a standard blister pack modified with conductive ink for transmitting data relating to blister pack stored medications; a blister pack tray adapted to receive a blister pack, and adapted to receive data transmitted from the blister pack; an end user computing device adapted to mechanically connect to the blister tray, whereby the data relating to blister pack stored medications is receivable to the computing device, and wherein the computing device runs software to identify changes in the status of blister pack stored medications; and a communications cloud, including a medication compliance tracking server and a medication compliance tracking database.

In some embodiments, the user device communicatively connects to the communications cloud, to enable dynamic order procurement of medications by the user.

In some embodiments, the user device communicatively connects to the communications cloud, to enable medication consumption data to be shared with other remote users, wherein remote user access medication consumption data requires user authentication.

In some embodiments, the user device communicatively connects to the communications cloud, to enable user health information to be shared between authorized remote users.

In some embodiments, the medications in the standard medication pack are maintained in cGMP-compliant packaging.

According to some embodiments of the present invention, a method is provided for managing oral medication consumption, that includes manufacturing a smart blister pack by printing a conductive circuit on a standard blister pack, wherein the smart blister pack is connectable to an end user computing device that tracks medication consumption from the smart blister pack.

In some embodiments, the method includes manufacturing a smart blister pack tray by printing a conductive circuit on a tray designed to hold a manufactured blister pack, wherein the smart blister pack holder is connectable to an end user computing device that tracks medication consumption from the smart blister pack holder.

In some embodiments, the method includes running software on the computing device, to process signals received from the conductive circuit, to determine extraction of one or more medication units from the smart blister pack holder.

In sonic embodiments, the method includes executing an algorithm on the computing device, to alert one or more users if one or more medication units are extracted from the smart blister pack holder.

In some embodiments, the method includes executing an algorithm on the computing device, to alert one or more users if one or more medication units are extracted from the smart blister pack holder in a way that contradicts a preprogrammed medication consumption protocol.

In some embodiments, the printing a conductive circuit on a standard blister pack maintains the authenticity of the original cGMP-compliant packaging.

According to some embodiments of the present invention, a device is provided for tracking oral medication consumption, that includes a blister pack tray with conductive ink adapted to receive a blister pack, and adapted to communicate data relating to blister pack stored medications transmitted to a connected end user computing device adapted to mechanically connect to the blister tray.

In sonic embodiments, the data relating to blister pack stored medications is receivable to the computing device, and wherein the computing device runs software to identify changes in the status of blister pack stored medications.

In some embodiments, the blister pack is modified with one or more layers of conductive ink for transmitting data relating to blister pack stored medications.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 is a schematic system diagram depicting components of a medication management and compliance system, according to some embodiments; and

FIGS. 2A-2D are graphic figures of blister packages, blister pack holders and an example of an associated blister management device, according to some embodiments;

FIGS. 3A-3D are schematic diagram depicting examples of system components designed to facilitate usage of blister packages with blister management devices, according to some embodiments;

FIGS. 4A-4C are graphic examples of blister packages adapted to be used in association with blister management devices, according to some embodiments;

FIG. 5 is a flow diagram depicting a process by which blister packages adapted to be used in association with blister management devices may be manufactured, according to some embodiments; and

FIGS. 6A-6C are screenshots or graphic simulations of examples of interfaces on communication devices.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

The term “blister pack” as used herein refers to pills packs, medication packs, blister cards, pill cards, medication containers, or other pre-formed packaging used for pharmaceuticals, for example, plastic, aluminum and/or paper based.

Non-limiting embodiments of the present invention include a system, device and methods for facilitating enhanced medication management and compliance management for substantially any blister packs, by adapting standard blister pack to be used in association with blister management devices, without interfering with the contents of such a blister pack, or the CGMP compliance packaging of medications.

In accordance with some embodiments, a method, device, and system are provided that enable objective measurement of a therapy compliance of patients who are undergoing a prescribed medication therapy.

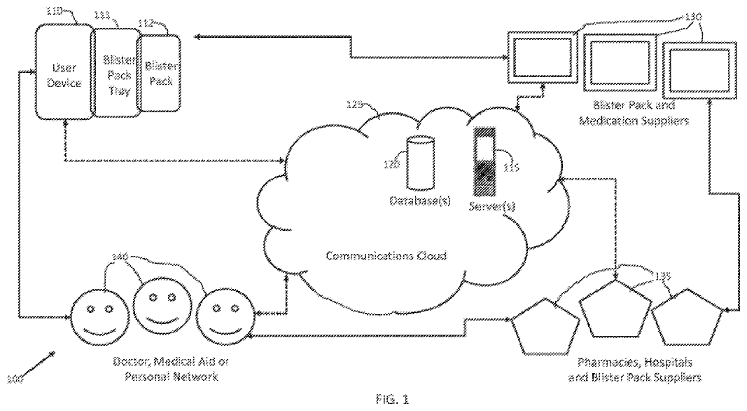

Reference is now made to FIG. 1, which is a schematic system diagram depicting a medication management and compliance system, according to some embodiments. As can be seen, the system includes a medication compliance management end user device 110, to be used by consumers of medications to manage and track medication consumption from substantially any blister packs. Device 110 is generally coupled to a blister pack tray or holder 111, that is designed to hold smart blister pack 112, and mechanically couple to user device 110. Device 110 is communicatively connected to a Server 115 and database 120, generally functioning within communications cloud 125, to provide processing, tracking, ordering, and other medication management functions to support blister pack management device usage. Blister pack medication manufacturers 130, for example, drug manufacturers, optionally manufacturing simple or multiple blister packs, may also be connected to communications cloud 125. Manufacturers 130 may integrate smart blister pack generation technology into their production of substantially any blister packs. Blister pack suppliers 135, for example, pharmacies, hospitals and other blister pack suppliers, may also be connected to communications cloud 125. Suppliers 135 may consolidate orders, prescriptions etc. and distribute medications, optionally in single or multiple blister packs. Suppliers 135 may integrate smart blister pack generation technology into their production of substantially any blister packs. Medical practitioners, professionals, doctors, medical aids, medical support or other network support people or systems 140 may also be connected to communications cloud 125. Practitioners 140 may prepare subscriptions and/or monitor or otherwise support medication compliance system.

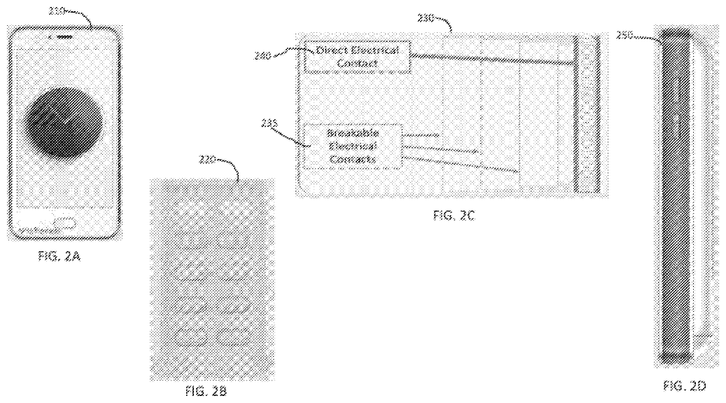

FIGS. 2A-2D are schematic and graphic figures of blister packages and associated blister management devices, according to some embodiments. As can be seen in FIG. 2A, a smartphone type communications and/or computing medication compliance end user management device 210 is provided. Device 210 may be a multipurpose device and/or a dedicated medication compliance management device. In some embodiments device 210 may include a means for patient identification, a means to query users about symptoms and other information, and radio transmission capability. In some embodiments device 210 is connected by wireless or wired electronic means to a remote computer that stores medical information about the user's medical condition, the user's treatment plan, the user's compliance with the treatment plan, and/or any recorded complications etc.

As can be seen in FIG. 2B, a blister pack loader 220 or connector apparatus is provided, for enabling substantially any blister pack to be coupled to compliance management device 210, Loader 220 is generally adapted to be coupled or attached to compliance management device 210, so as to enable the loaded blister pack (230) to be communicatively connected to compliance management device 210. Loader 220 may be adapted to function as a handheld medication dispenser capable of receiving and storing medication blister packs, for example, blister packs that are manufactured using standard pharmaceutical packaging equipment.

As can be seen in FIG. 2C, a schematic example of a smart blister pack 230 is shown, which includes a substantially standard blister pack with selected breakable electrical contacts or tracks 235, and direct electrical contacts 240. In some embodiments, a conductive circuit is printed on a substrate, characterized in that the conductive tracks function as signal emitters for an IC, antenna and any other components in some embodiments, a polymer-based electronic circuit is printed directly on a blister film or a foil, using conductive printing, referred to herein as a system-in-foil or system-on-foil technology. In one embodiment, the printed polymer circuit is used to identify the identity of the medication. In another embodiment, the printed polymer circuit is used to track status changes of medications in a blister pack. For example, where a number of conductive tracks are used for running under each blister, when a pill is expelled from a blister thereby breaking the foil seal, the corresponding track is broken, thereby emitting a signal or ceasing an existing signal, showing this change. Tracks 235 are generally connected to a silicon or printed polymer integrated circuit, for example, contacts 240, that record the blister status change events, for example when a track is broken, and transmits this information, together with the identity of the user, in real time to the management device. This data may be further processed by the management device. The management device may optionally transmit this data and/or processed data using wireless radio technology, including the time of the event and the position of the blister and any other clinically relevant data.

In some embodiments, a light-emitting system and photoreceptor system is used to trans-illuminate the blister pack and identify that a blister has been broken. In this embodiment, the light transmission apparatus (e.g., photo array) may be connected to a silicon or printed polymer integrated circuit, as is the light-receiving apparatus (e.g., photo sensor) that records the event when the seal is broken and stores this information, including the time of the event and the position of the blister and any other clinically relevant data. The electronic circuit may also contain electrical contacts, enabling its state to be read by a reader, for example, integrated into remote computing device,

In some embodiments, the integrated circuit of the smart blister pack can be programmed using an RF1D interface during the blister-filling phase with information about the tilling medication, for example, including unique codes to counteract counterfeiting, and logistical data.

In some embodiments, the integrated circuit of the smart blister pack may further include an antenna and a power source, both of which may be printed during the same, or a separate, system-in-foil and/or system-on-foil process, and/or mounted as discrete components. The intelligent system-in-foil or system-on-foil elements, in accordance with some embodiments, may replace the standard film or foil currently used in the existing standard blister packaging manufacturing process.

FIG. 2D shows a graphic example of a blister pack loader 220 coupled to a compliance management device 210. Of course, other formats, forms, shapes and designs may be used for the blister pack loader 220 and the compliance management device 210. In some embodiments, current for reading medication status data in a smart blister pack is drawn from the battery of device 210, through electrical contacts to the conductive layer of blister pack 220. Device 210 may recognize every flow of current that returns to the device, and thereby identify every trace that has been broken, indicating that a medication has been consumed.

In some embodiments, compliance management device 210 may be a communications device 250, for example, a cellular enabled device, optionally with a S1M. Device 210 may further include multiple monitors or sensors via connected ports, whether wire or wireless. Further, compliance management device 210 may generally run software, code or an Application to manage medication compliance, for example, by tracking and processing medication consumption. For example, compliance management device 210 may run files with instructions to execute commands to enable determination if a blister pack is coupled to the device, what a connected blister pack contains, the dosage of any medication detected, which medications in a connected pack have been consumed, the authenticity of the medication and medication packaging, and more advanced medication procurement, tracking and ordering functions. Device 210 may further include a user Interface, for example, a touch screen with a Graphical User Interface (GUI) representing the medication related data being managed. In further embodiments, compliance related data is processed in compliance device. In further embodiments, device 210 may be communicatively connected to a communications cloud, wherein a compliance management system server(s) and database(s) are running. In some embodiments, usage of a communications cloud ecosystem may allow for multiple user cooperation, reporting, alerting, monitoring, data backup, ordering, and more.

In some embodiments, compliance management device 210 is adapted to identify the change in state of the medication blister pack whenever a medication dose is removed.

In some embodiments, compliance management device 210 is adapted to identify the identity of one or more medications contained in the blister pack.

In some embodiments, compliance management device 210 is adapted to identify a touch-screen display designed to impart information to the user and collect information from the user.

In some embodiments, compliance management device 210 includes a radio connection to a remote computer which contains patient information and treatment algorithms.

In some embodiments, compliance management device 210 includes means of securing user identification, to help prevent unauthorized use or diversion of the tracked medication.

In some embodiments, compliance management device 210 is adapted to execute treatment algorithms for the specific user.

In some embodiments, compliance management device 210 is adapted to execute algorithms to track the behavior of a blister pack user, thereby aiding measurement of medication.

In some embodiments, compliance management device 210 is adapted to execute algorithms to effect notifications or alerts to other parties responsible for or associated with the user's care. For example, after identifying a deviation from a treatment protocol, the device 210 may generate an alerts that is sent to one or more parties listed in a remote database, for example, medical personnel, and designated friends and/or family,

In some embodiments, compliance management device 210 includes means to verify a user's identity, for example, using fingerprint recognition, facial recognition, voice recognition, and/or a secure password.

In some embodiments, compliance management device 210 may include ancillary sensors to measure physiologic parameters including but not limited to: pulse, blood pressure, blood glucose or other analytes, heart rhythm, body temperature, breath volume, and other such parameters may be attached to the receiver through wired or wireless connections.

In some embodiments, compliance management device 210 may transmit directly to a remote computer in order to monitor effects of medication use or compliance with medication use.

In some embodiments, compliance management device 210 includes electromechanical locking mechanisms incorporated into the handheld receiver in order to regulate use of medication according to a treatment algorithm.

In some embodiments, compliance management device 210 includes means for preventing erroneous medication consumption. For example, if a mistake is noted in a user action, such as removing an incorrect product, or removing a product at the wrong time, a light, acoustic error signal and/or an error message may be displayed on or generated by the device. In other cases, a radio message may be transmitted, for example in the form of a text message, listing the error and advising the user or other related followers, to take alternative action. In some examples artificial intelligence or other suitable algorithms may be run to predict or identify deviations from preprogrammed protocols.

In some embodiments, blister pack loader 220 may function as a smart blister package including one or more medications and/or types of medications, that includes a conductive ink circuit for transmitting medication status data between blister package 230 and compliance management device 210. In this way, usage of conductive ink functionally turns blister package 230 into a circuit board, allowing substantially any blister pack to be made into a smart pack to enable communication with a management device. In further embodiments, blister pack 230 may use MID, Bluetooth or other wireless technologies to transmit data to the management device. In some embodiments, conductive ink may be selectively applied to blister pack 230, to track pill consumption, serialize medication etc. In some embodiments, conductive ink may be selectively applied to blister pack 230 in such a way that the packaged pills or medications are not touched while being marked or serialized.

In some embodiments, the blister pack may be used to store multiple products, whereby each product has a designated location, whereby a passive electrical circuit is associated with each designated location, that can be disrupted under specific conditions. In this way, if the status of a product in a dedicated location changes, this may stop the conducting of an electrical signal along the relevant conductive tracks that lead from the dedicated location to a sensor. In some embodiments, such tracks may be formed by electrically conductive material such as metal tracks, tracks of conductive ink or of conductive polymers, that are printed on a non-conductive substrate in order to have no electrical connection to the aluminum or other metallic foil of the blister pack.

In some embodiments, each blister pack has a defined pattern that may be printed on a sealing foil, which seals the blister pack compartments, and that includes the defined pattern and the components, that are printed on it, for example using contact OW or a dedicated laminating process.

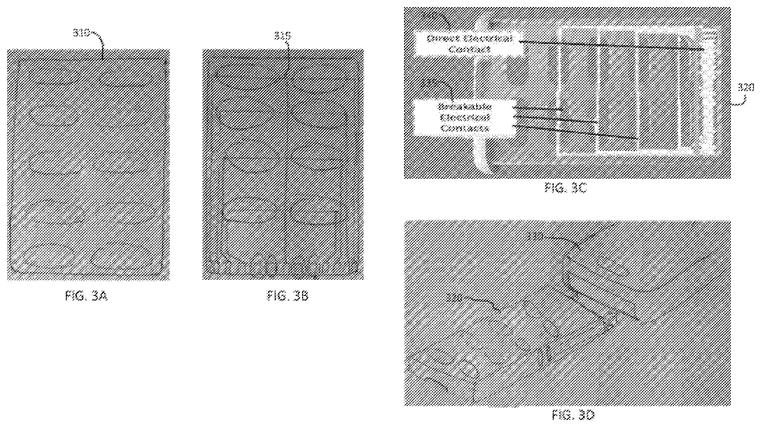

FIGS. 3A-3B are graphic examples of conductive layers of blister packages and a conductive layers of a blister packages holder, adapted to be used in association with blister management devices, according to some embodiments. As can be seen in FIG. 3A, layer 1 of a blister pack may include a standard blister package 310 may be used to provide medications to a user. Multiple medications and/or types of medications may be used. For example, a fully sealed thermoformed medication package or vacuum pack may be used.

As can be seen in FIG. 3B, layer 2 of a blister pack may include conductive ink 315 applied to blister package 310, to enable information about medications in blister pack 310 to be transmitted to a compliance management device (330). In some embodiments the conductive layer may be applied using inkjet printing. In some embodiments the conductive layer may be applied using silk screening. For example, elements may be printed by industrial inkjet printers, or silk screening, that lay down ink optionally based on pre-set patterns. For example, such patterns are input by printing software according to the medication and/or layout of the blister pack. Of course, other printing or applying methods may be used. In some embodiments, the conductive layer is based on special conductive inks. In some applications, these inks are constructed carbon, copper and/or silver. In some embodiments, after the ink has been applied via industrial inkjet printers or a silk screening process, it may be sintered, thereby heating the metals to below their boiling point, to harden and stabilize the ink. In some cases, the sintering is performed in a traditional oven format, where the printed material is baked over time. In other cases, high-powered Infra-Red, or other heat or radiation sources may be used in further embodiments, a conductive layer may be applied that has a resistance of any variety. In still further embodiments, an extensive serialization may be enabled by including additional electrical contacts, by changing resistive properties of the conductive ink, and/or by changing additional active properties of the conductive ink. In some embodiments, such extended serialization, optionally including variations in electrical properties, may enable more or higher resolution data to be tracked and/or transmitted from a blister pack or series of packs.

FIG. 3C is a graphic example of a conductive layers of a blister package holder, adapted to be used in association with blister management devices, according to some embodiments. In some embodiments, a conductive layer or layers may be added to the holder apparatus or tray 320, for example, using breakable plastic, to allow for easy pill removal past the breakable conductive trace, wherein the conductive ink may be printed directly on the back of the blister packs and/or onto an intermediary layer and adhered to either side. In some embodiments, a breakable conductive layer may be located below the blister pack, such that each conductive line or trace 335 must break as the pill passes out of its opening, and that the conductive traces must follow back to a grid of electrical contacts 340 that are read by a complimentary contact inside the user compliance device.

FIG. 3D is a graphic example of a blister package holder being coupled with a blister management device, according to some embodiments. As can be seen in FIG. 3D, blister pack holder 320 may include a tray adapted to mechanically connect or couple to a compliance management device 330. In some embodiments, apparatus 320 may be hard tray for placement in a management device, or on a management device. In some embodiments, a conductive layer may be added to the holder apparatus or tray, for example, using breakable plastic, wherein the conductive ink is printed directly on the back of the blister packs and/or onto an intermediary layer and adhered to either side.

In some embodiments, in order to fix a conductive layer to substantially any blister pack, one or more of the following options may be applied: Printing the conductive material onto the foil of the blister pack; Printing the conductive layer onto the hard plastic tray to which the blister pack is coupled, in which case such a tray will require perforation or scoring to allow for easy breaking from the package; and/or by adding a third layer between the blister pack and the tray with similar perforation or scoring, which may allow for usage of an all-purpose tray that can be coupled to the blister pack and tray.

In some examples, blister packaging may be produced using a pre-formed press to line up the pills and seal them in order. Once the pattern is known for a press, set patterns may be generated for each medication, or even for each press itself

In some examples, a packaging or distribution entity can package medication with such smart blister packaging using the above described method, and distribute to pharmacy or other networks.

In some embodiments, once a blister pack holder is inserted into the management device, the device will read the conductive layer for serialization data, for example, reading the serial number of the blister pack and/or the medications that are in the blister pack. In the case where a serial number of a medication package is provided, this serial number may indicate the composition or contents of the blister pack, which can then be read by the management device.

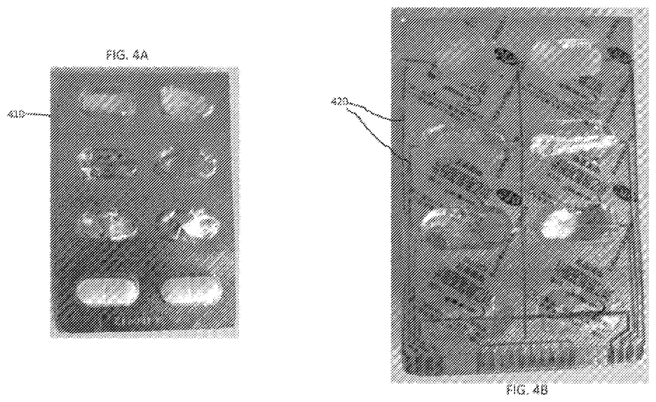

FIGS. 4A-4B are graphic examples of blister packages adapted to be used in association with blister management devices, according to some embodiments. As can be seen in FIG. 4A, a standard blister pack 410 is shown, that includes 8 pills in blister type compartments. As can be seen in FIG. 4B, conductive ink 420 may be printed on the back on blister pack 410, such that the conductive ink crosses over the breakable aluminum cover of each compartment (i.e., breakable electrical contacts), such that the management device will “know” when each compartment is closed (unused) or open (used). As can be seen, blister package 410 may include printed direct electrical contacts, for providing a point of contact between the blister pack and the blister pack holder apparatus.

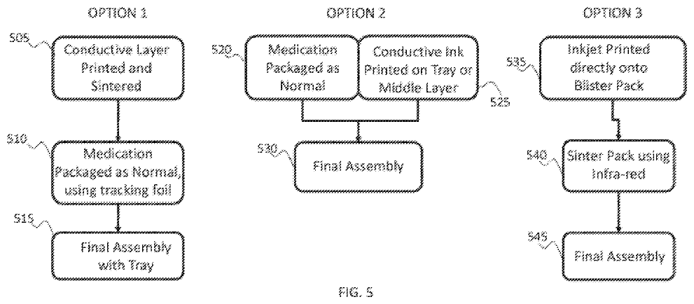

FIG. 5 is a flow diagram depicting a process by which blister packages adapted to be used in association with blister management devices may be manufactured/configured., according to some embodiments. As can be seen, there are several options that may be applied to produce a smart blister package that may be read by a compliance management device, as described above. In some embodiments the conductive ink may be applied to the blister packs on the production line, whether on the packaged pill packs or covers, on an intermediary layer, and/or on the blister pack holding trays. In some embodiments the conductive ink layer may be designed by a computer program, for example, using CAD or other software programs, detailing how and where it is to be applied in accordance with the serial number or detailed description of the blister pack design/content etc. In some embodiments additional system components to add conductive ink may be provided, for example, during or following blister pack production. For example, an ink jet printer and/or silk screening machinery may be used as part of production or post production of blister packs, to print conductive ink to a blister pack. Further, a sintering means may be used as part of production or post production of blister packs, to appropriately heat the conductive ink as may be required, in some cases, a rapid sintering process may be applied. Further, additional assembling or packaging means may be used to couple a blister pack or multiple blister packs to one or more holders or trays.

As can be seen, for Option 1, at step 505 a conductive layer is printed and sintered on a blister pack, during production. At step 510, the medication is packed as usual, but attaching a customized tracking foil that includes the relevant conductive traces and electrical contacts. At step 515 the final assembling is implemented, where the blister packs are packaged and distributed with a smart blister pack holder or tray.

As can be seen, for Option 2, at step 520 a medication package is packed as normal. At step 525 a conductive layer is printed on a blister pack tray and/or an intermediate or middle layer. At step 530, the final assembling is implemented, where the blister packs are packaged and distributed with a smart blister pack holder or tray.

As can be seen, for Option 3, at step 535 a conductive layer is printed using an Inkjet printer, onto a blister pack, during production. At step 540, the blister pack is sintered, optionally using infra-red radiation. At step 545 the final assembling is implemented, where the blister packs are combined with (hard plastic) trays and distributed.

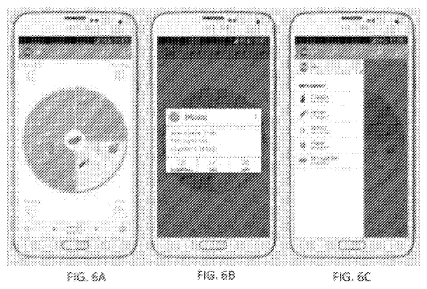

FIGS. 6A-6C are screenshots or graphic simulations of three examples of interfaces on compliance management devices. As can be seen, in FIG. 6A, a GUI is provided with an inner medication tracking scheduler and tracker, to help a user easily understand which medications they need to take in a given period, and which are taken and which are not. As can be seen, in FIG. 6B, the GUI includes a pop up box that aids the user with medication schedule information, and enabled rapid entry to confirm consumption of the medication. As can be seen, in FIG. 6C, the GUI includes a pop up that shows the user the available medications in a connected blister pack or blister pack holder. In this example, the dynamic pop up will change to reflect the status of the medications in the connected blister pack. Of course, other GUI functions, forms and designs may be used to facilitate blister pack management and medication compliance.

In some embodiments, the blister pack holder may include additional meters or sensors. In one example, a blood glucose meter and relevant sensors may be included or coupled to a blister pack holder.

In some embodiments the blister pack holder may include one or more biometrics sensors, for example, to help enable user identification, tracking, safety and biosecurity.

In some embodiments the blister pack holder may include a locking mechanism.

In some embodiments a method for measuring and monitoring an extraction of a correct medication at a correct time is provided, that enables observers to improve supervision of a therapy compliance of a patient, immediately or at a later stage.

In some embodiments the system, device and methods for facilitating enhanced medication management and compliance management may be used to support clinical trials compliance and results monitoring.

In some embodiments the system, device and methods for facilitating enhanced medication management and compliance management may be used to track medication diversion, abuse, expiration and/or counterfeiting.

In some embodiments the blister pack holder may transmit to the compliance management device medicine management data, for example, chronological data to determine the order in which blister packs are to be used. In further embodiments, the blister pack holder may transmit to the compliance management device medicine management data, for example, to help users with ordering and/or delivery of user medications, based on medication usage. In further embodiments, the blister pack holder may transmit to the compliance management device medicine management data, for example, to help a user understand what medication has been provided, information about the medication, safety information etc.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

What is claimed is:

1. A system for tracking medication compliance, comprising:
a blister pack modified with conductive ink for transmitting data relating to medications stored in the blister pack;
a blister pack tray adapted to receive the blister pack, and adapted to receive data transmitted from the blister pack;
an end user computing device adapted to mechanically connect to the blister pack tray, whereby the end user computing device is configured to receive data relating to medications stored in the blister pack, and wherein the end user computing device is configured to run software to identify changes in the status of medications stored in the blister pack; and
a communications cloud, including a medication compliance tracking server and a medication compliance tracking database.

2. The system of claim 1, wherein the end user computing device communicatively connects to the communications cloud, to enable dynamic order procurement of medications by the user.

3. The system of claim 1, wherein the end user computing device communicatively connects to the communications cloud, to enable medication consumption data to be shared with other remote users, wherein remote user access medication consumption data requires user authentication.

4. The system of claim 1, wherein the end user computing device communicatively connects to the communications cloud, to enable user health information to be shared between authorized remote users.

5. The system of claim 1, wherein the medications stored in the blister pack are maintained in cGMP-compliant packaging.

6. A method of managing oral medication consumption, comprising: manufacturing a smart blister pack by printing a conductive circuit on a blister pack, wherein the smart blister pack is connectable to an end user computing device that tracks medication consumption from the smart blister pack.

7. The method of claim 6, further comprising manufacturing a smart blister pack tray by printing a conductive circuit on a tray configured to hold a blister pack, wherein the smart blister pack holder is connectable to an end user computing device that tracks medication consumption from the smart blister pack holder.

8. The method of claim 6, further comprising running software on the end user computing device, to process signals received from the conductive circuit, to determine extraction of one or more medication units from the smart blister pack.

9. The method of claim 6, comprising executing an algorithm on the end user computing device, to alert one or more users if one or more medication units are extracted from the smart blister pack holder.

10. The method of claim 6, comprising executing an algorithm on the end user computing device, to alert one or more users if one or more medication units are extracted from the smart blister pack holder in a way that contradicts a preprogrammed medication consumption protocol.

11. The method of claim 6, whereby the printing a conductive circuit on a blister pack maintains the authenticity of the original cGMP-compliant packaging.

12. A device for tracking oral medication consumption, comprising: a blister pack tray with conductive ink adapted to receive a blister pack, and adapted to communicate data relating to medications stored in the blister pack to a connected end user computing device adapted to mechanically connect to the blister tray.

13. The device of claim 12, whereby the data relating to the medications stored in the blister pack is receivable to the end user computing device, and wherein the end user computing device runs software to identify changes in the status of the medications stored in the blister pack.

14. The device of claim 12, wherein the blister pack includes one or more layers of conductive ink configured to transmit data relating to medications stored in the blister pack.
